(19) United States
(12) Patent Application Publication
(10) Pub. No.: US 2012/0308507 A1
(43) Pub. Date: Dec. 6, 2012

(54) Odor-Inhibiting Mixtures for Incontinence Articles

(75) Inventors: Asif Karim, Mannheim (DE); Thomas Daniel, Waldsee (DE)

(73) Assignee: BASF SE, Ludwingshafen (DE)

(21) Appl. No.: 13/482,450

(22) Filed: May 29, 2012

Related U.S. Application Data
(60) Provisional application No. 61/491,916, filed on Jun. 1, 2011.

(51) Int. Cl. A61L 11/00

(52) U.S. Cl. 424/76.6

(57) ABSTRACT

Odor-inhibiting mixtures comprising water-absorbing polymer particles and spherical activated carbon for use in incontinence articles.

The present invention relates to odor-inhibiting mixtures comprising water-absorbing polymer particles and spherical activated carbon for use in incontinence articles.

Water-absorbing polymer particles are used to produce diapers, tampons, sanitary napkins and other hygiene articles, but also as water-retaining agents in market gardening. The water-absorbing polymer particles are also referred to as superabsorbents.

The production of water-absorbing polymer particles is described in the monograph “Modern Superabsorbent Polymer Technology”, F. L. Buchholz and A. T. Graham, Wiley-VCH, 1998, pages 71 to 103.

The properties of the water-absorbing polymer particles can be adjusted, for example, via the amount of crosslinker used. With increasing amount of crosslinker, the centrifuge retention capacity (CRC) falls and the absorption under a pressure of 21.0 g/cm² (AUL0.3 psi) passes through a maximum.

To improve the use properties, for example, permeability of the swollen gel bed (SFC) in the diaper and absorption under a pressure of 49.2 g/cm² (AUL0.7 psi), water-absorbing polymer particles are generally surface postcrosslinked. This increases the crosslinking of the particle surface, which can at least partly decouple the absorption under a pressure of 49.2 g/cm² (AUL0.7 psi) and the centrifuge retention capacity (CRC). This surface postcrosslinking can be performed in aqueous gel phase. Preferably, however, dried, ground and sieved polymer particles (base polymer) are surface coated with a surface postcrosslinker and thermally surface postcrosslinked. Crosslinkers suitable for that purpose are compounds which can form covalent bonds to at least two carboxylate groups of the water-absorbing polymer particles.

There has also been no lack of attempts to prevent the occurrence of unpleasant odors in the course of use of hygiene articles. According to US 2008/0179 A1, US 2008/0147028 A1, US 2010/0286645 A1, EP 1 358 894 A1 and WO 98/26808 A2, it is possible to use activated carbon for this purpose.

It was an object of the present invention to provide improved odor-inhibiting mixtures for use in incontinence articles. The mixtures should especially have a relatively high whiteness.

In addition, the plant parts used for production of the odor-inhibiting mixtures should be easy to clean, such that contamination of the next production campaign is prevented in the event of product changes.

The object was achieved by odor-inhibiting mixtures comprising water-absorbing polymer particles and spherical activated carbon.

Activated carbon is typically used in the form of powder, crushed particles or compressed rods. The inventive spherical activated carbons, in contrast, are substantially monodisperse spheres.

The water-absorbing polymer particles are obtained, for example, by polymerizing a monomer solution or suspension comprising:

a) at least one ethylenically unsaturated monomer which bears acid groups and may be at least partly neutralized,

b) at least one crosslinker,

c) at least one initiator,

d) optionally one or more ethylenically unsaturated monomers copolymerizable with the monomers mentioned under a) and

e) optionally one or more water-soluble polymers,

and are typically water-insoluble.

The monomers a) are preferably water-soluble, i.e. the solubility in water at 23° C. is typically at least 1 g/100 g of water, preferably at least 5 g/100 g of water, more preferably at least 25 g/100 g of water and most preferably at least 35 g/100 g of water.

Suitable monomers a) are, for example, ethylenically unsaturated carboxylic acids, such as acrylic acid, methacrylic acid and itaconic acid. Particularly preferred monomers are acrylic acid and methacrylic acid. Very particular preference is given to acrylic acid.

Further suitable monomers a) are, for example, ethylenically unsaturated sulfonic acids, such as styrenesulfonic acid and 2-acrylamido-2-methylpropanesulfonic acid (AMPS).

Impurities can have a considerable influence on the polymerization. The raw materials used should therefore have a maximum purity. It is therefore often advantageous to specially purify the monomers a). Suitable purification processes are described, for example, in WO 2002/055469 A1, WO 2003/078378 A1 and WO 2004/035514 A1. A suitable monomer a) is, for example, acrylic acid purified according to WO 2004/035514 A1 and comprising 99.8460% by weight of acrylic acid, 0.0950% by weight of acetic acid, 0.0332% by weight of water, 0.0203% by weight of propionic acid, 0.0001% by weight of furfurals, 0.0001% by weight of maleic anhydride, 0.0003% by weight of diacrylic acid and 0.0050% by weight of hydroquinone monomethyl ether.

The proportion of acrylic acid and/or salts thereof in the total amount of monomers is preferably at least 50 mol %, more preferably at least 90 mol %, most preferably at least 95 mol %.

The acrylic acid used typically comprises polymerization inhibitors, preferably hydroquinone monoethers, as storage stabilizers.

The monomer solution therefore comprises preferably up to 250 ppm by weight, preferably at most 130 ppm by weight, more preferably at most 70 ppm by weight, and preferably at least 10 ppm by weight, more preferably at least 30 ppm by weight and especially around 50 ppm by weight, of hydroquinone monoether, based in each case on the unneutralized acrylic acid. For example, the monomer solution can be prepared using an acrylic acid with an appropriate hydroquinone monoether content.

Preferred hydroquinone monoethers are hydroquinone monomethyl ether (MEHQ) and/or alpha-tocopherol (vitamin E).

Suitable crosslinkers b) are compounds having at least two groups suitable for crosslinking. Such groups are, for example, ethylenically unsaturated groups which can be polymerized free-radically into the polymer chain, and functional groups which can form covalent bonds with the acid groups of the acrylic acid. In addition, polyvalent metal salts which can form coordinate bonds with at least two acid groups of the acrylic acid are also suitable as crosslinkers b).

Crosslinkers b) are preferably compounds having at least two polymerizable groups which can be polymerized free-radically into the polymer network. Suitable crosslinkers b) are, for example, ethylene glycol dimethacrylate, diethylene glycol diacrylate, polyethylene glycol diacrylate, allyl methacrylate, trimethylolpropane triacrylate, triallylamine, tetraallylammonium chloride, tetraallyloxyethane, as described in EP 0 530 438 A1, di- and triacrylates, as described in EP 0 547 847 A1, EP 0 559 476 A1, EP 0 632 068 A1, WO 93/21237 A1, WO 2003/104299 A1, WO 2003/104300 A1, WO 2003/104301 A1 and DE 103 31 450 A1, mixed acrylates which, as well as acrylate groups, comprise further ethylenically unsaturated groups, as described in DE 103 31 456 A1 and DE 103 55 401 A1, or crosslinker mixtures, as described, for example, in DE 195 43 368 A1, DE 196 46 484 A1, WO 90/15830 A1 and WO 2002/032962 A2.

Preferred crosslinkers b) are pentaerythrityl triallyl ether, tetraallyloxyethane, methylenebismethacrylamide, 15-tuply ethoxylated trimethylolpropane triacrylate, polyethylene glycol diacrylate, trimethylolpropane triacrylate and triallylamine.

Very particularly preferred crosslinkers b) are the polyethoxylated and/or -propoxylated glycerols which have been esterified with acrylic acid or methacrylic acid to give di- or triacrylates, as described, for example, in WO 2003/104301 A1. Di- and/or triacrylates of 3- to 10-tuply ethoxylated glycerol are particularly advantageous. Very particular preference is given to di- or triacrylates of 1- to 5-tuply ethoxylated and/or propoxylated glycerol. Most preferred are the triacrylates of 3- to 5-tuply ethoxylated and/or propoxylated glycerol, especially the triacrylate of 3-tuply ethoxylated glycerol.

The amount of crosslinker b) is preferably 0.05 to 1.5% by weight, more preferably 0.1 to 1% by weight and most preferably 0.2 to 0.6% by weight, based in each case on acrylic acid. With rising crosslinker content, the centrifuge retention capacity (CRC) falls and the absorption under a pressure of 21.0 g/cm² passes through a maximum.

The initiators c) used may be all compounds which generate free radicals under the polymerization conditions, for example thermal initiators, redox initiators, photoinitiators. Suitable redox initiators are sodium peroxodisulfate/ascorbic acid, hydrogen peroxide/ascorbic acid, sodium peroxodisulfate/sodium bisulfite and hydrogen peroxide/sodium bisulfite. Preference is given to using mixtures of thermal initiators and redox initiators, such as sodium peroxodisulfate/hydrogen peroxide/ascorbic acid. The reducing component used is, however, preferably a mixture of the sodium salt of 2-hydroxy-2-sulfinatoacetic acid, the disodium salt of 2-hydroxy-2-sulfonatoacetic acid and sodium bisulfite. Such mixtures are obtainable as Brüggolite® FF6 and Brüggolite® FF7 (Brüggemann Chemicals; Heilbronn; Germany).

Ethylenically unsaturated monomers d) copolymerizable with acrylic acid are, for example, acrylamide, methacrylamide, hydroxyethyl acrylate, hydroxyethyl methacrylate, dimethylaminoethyl methacrylate, dimethylaminoethyl acrylate, dimethylaminopropyl acrylate, diethylaminopropyl acrylate, dimethylaminoethyl methacrylate, diethylaminoethyl methacrylate.

The water-soluble polymers e) used may be polyvinyl alcohol, polyvinylpyrrolidone, starch, starch derivatives, modified cellulose, such as methylcellulose or hydroxyethylcellulose, gelatin, polyglycols or polyacrylic acids, preferably starch, starch derivatives and modified cellulose.

Typically, an aqueous monomer solution is used. The water content of the monomer solution is preferably from 40 to 75% by weight, more preferably from 45 to 70% by weight and most preferably from 50 to 65% by weight. It is also possible to use monomer suspensions, i.e. monomer solutions with excess acrylic acid, for example sodium acrylate. With rising water content, the energy expenditure in the subsequent drying rises, and, with falling water content, the heat of polymerization can be removed only inadequately.

For optimal action, the preferred polymerization inhibitors require dissolved oxygen. The monomer solution can therefore be freed of dissolved oxygen before the polymerization by inertization, i.e. flowing an inert gas through, preferably nitrogen or carbon dioxide. The oxygen content of the monomer solution is preferably lowered before the polymerization to less than 1 ppm by weight, more preferably to less than 0.5 ppm by weight, most preferably to less than 0.1 ppm by weight.

Suitable reactors are, for example, kneading reactors or belt reactors. In the kneader, the polymer gel formed in the polymerization of an aqueous monomer solution or suspension is comminuted continuously by, for example, contrarotatory stirrer shafts, as described in WO 2001/038402 A1. Polymerization on the belt is described, for example, in DE 38 25 366 A1 and U.S. Pat. No. 6,241,928. Polymerization in a belt reactor forms a polymer gel which has to be comminuted in a further process step, for example in an extruder or kneader.

To improve the drying properties, the comminuted polymer gel obtained by means of a kneader can additionally be extruded.

The acid groups of the resulting polymer gels have typically been partially neutralized. Neutralization is preferably carried out at the monomer stage. This is typically accomplished by mixing in the neutralizing agent as an aqueous solution or preferably also as a solid. The degree of neutralization is preferably from 25 to 95 mol %, more preferably from 30 to 80 mol % and most preferably from 40 to 75 mol %, for which the customary neutralizing agents can be used, preferably alkali metal hydroxides, alkali metal oxides, alkali metal carbonates or alkali metal hydrogencarbonates and also mixtures thereof. Instead of alkali metal salts, it is also possible to use ammonium salts. Particularly preferred alkali metals are sodium and potassium, but very particular preference is given to sodium hydroxide, sodium carbonate or sodium hydrogencarbonate and also mixtures thereof.

However, it is also possible to carry out neutralization after the polymerization, at the stage of the polymer gel formed in the polymerization. It is also possible to neutralize up to 40 mol %, preferably from 10 to 30 mol % and more preferably from 15 to 25 mol % of the acid groups before the polymerization by adding a portion of the neutralizing agent actually to the monomer solution and setting the desired final degree of neutralization only after the polymerization, at the polymer gel stage. When the polymer gel is neutralized at least partly after the polymerization, the polymer gel is preferably comminuted mechanically, for example by means of an extruder, in which case the neutralizing agent can be sprayed, sprinkled or poured on and then carefully mixed in. To this end, the gel mass obtained can be repeatedly extruded for homogenization.

The polymer gel is then preferably dried with a belt drier until the residual moisture content is preferably 0.5 to 15% by weight, more preferably 1 to 10% by weight and most preferably 2 to 8% by weight, the residual moisture content being determined by EDANA recommended test method No. WSP 230.2-05 “Mass Loss Upon Heating”. In the case of too high a residual moisture content, the dried polymer gel has too low a glass transition temperature T_{g} and can be processed further only with difficulty. In the case of too low a residual moisture content, the dried polymer gel is too brittle and, in the subsequent comminution steps, undesirably large amounts of polymer particles with an excessively low particle size are obtained (“fines”). The solids content of the gel before the drying is preferably from 25 to 90% by weight, more preferably from 35 to 70% by weight and most preferably from 40 to 60% by weight. However, a fluidized bed drier or a paddle drier may optionally also be used for drying purposes.

Thereafter, the dried polymer gel is ground and classified, and the apparatus used for grinding may typically be single or multistage roll mills, preferably two or three-stage roll mills, pin mills, hammer mills or vibratory mills.

In a preferred embodiment of the present invention, an aqueous monomer solution is dropletized and the droplets obtained are polymerized in a heated carrier gas stream. It is possible here to combine the process steps of polymerization and drying, as described in WO 2008/040715 A2, WO 2008/052971 A1 and especially in WO 2011/026876 A1. In this preferred embodiment, the particle size is adjusted via the size of the droplets obtained.

The mean particle size of the water-absorbing polymer particles is preferably at least 200 μm, more preferably from 250 to 600 μm and very particularly from 300 to 500 μm. The mean particle size can be determined by means of EDANA recommended test method No. WSP 220.2-05 “Particle Size Distribution”, where the proportions by mass of the screen fractions are plotted in cumulated form and the mean particle size is determined graphically. The mean particle size here is the value of the mesh size which gives rise to a cumulative 50% by weight.

The proportion of particles with a particle size of greater than 150 μm is preferably at least 90% by weight, more preferably at least 95% by weight, most preferably at least 98% by weight.

Polymer particles with too small a particle size lower the permeability (SFC). The proportion of excessively small polymer particles (“fines”) should therefore be low.

Excessively small polymer particles are therefore typically removed and recycled into the process. This is preferably accomplished before, during or immediately after the polymerization, i.e. before the drying of the polymer gel. The excessively small polymer particles can be moistened with water and/or aqueous surfactant before or during the recycling.

It is also possible to remove excessively small polymer particles in later process steps, for example after the surface postcrosslinking or another coating step. In this case, the excessively small polymer particles recycled are surface postcrosslinked or coated in another way, for example with fumed silica.

When a kneading reactor is used for polymerization, the excessively small polymer particles are preferably added during the last third of the polymerization.

When the excessively small polymer particles are added at a very early stage, for example actually to the monomer solution, this lowers the centrifuge retention capacity (CRC) of the resulting water-absorbing polymer particles. However, this can be compensated for, for example, by adjusting the amount of crosslinker b) used.

When the excessively small polymer particles are added at a very late stage, for example not until an apparatus connected downstream of the polymerization reactor, for example an extruder, the excessively small polymer particles can be incorporated into the resulting polymer gel only with difficulty. Insufficiently incorporated, excessively small polymer particles are, however, detached again from the dried polymer gel during the grinding, are therefore removed again in the course of classification and increase the amount of excessively small polymer particles to be recycled.

The proportion of particles having a particle size of at most 850 μm is preferably at least 90% by weight, more preferably at least 95% by weight, most preferably at least 98% by weight.

The proportion of particles having a particle size of 150 to 850 μm is preferably at least 90% by weight, more preferably at least 95% by weight, most preferably at least 98% by weight.

Polymer particles of excessively large particle size lower the free swell rate. The proportion of excessively large polymer particles should therefore likewise be small.

Excessively large polymer particles are therefore typically removed and recycled into the grinding of the dried polymer gel.

To further improve the properties, the polymer particles can be surface postcrosslinked. Suitable surface postcrosslinkers are compounds which comprise groups which can form covalent bonds with at least two carboxylate groups of the polymer particles. Suitable compounds are, for example, polyfunctional amines, polyfunctional amido amines, polyfunctional epoxides, as described in EP 0 083 022 A2, EP 0 543 303 A1 and EP 0 937 736 A2, di- or polyfunctional alcohols, as described in DE 33 14 019 A1, DE 35 23 617 A1 and EP 0 450 922 A2, or β-hydroxyalkylamides, as described in DE 102 04 938 A1 and U.S. Pat. No. 6,239,230.

Additionally described as suitable surface postcrosslinkers are cyclic carbonates in DE 40 20 780 C1, 2-oxazolidinone and derivatives thereof, such as 2-hydroxyethyl-2-oxazolidinone, in DE 198 07 502 A1, bis- and poly-2-oxazolidinones in DE 198 07 992 C1, 2-oxotetrahydro-1,3-oxazine and derivatives thereof in DE 198 54 573 A1, N-acyl-2-oxazolidinones in DE 198 54 574 A1, cyclic ureas in DE 102 04 937 A1, bicyclic amide acetals in DE 103 34 584 A1, oxetanes and cyclic ureas in EP 1 199 327 A2 and morpholine-2,3-dione and derivatives thereof in WO 2003/031482 A1.

Preferred surface postcrosslinkers are ethylene carbonate, ethylene glycol diglycidyl ether, reaction products of polyamides with epichlorohydrin and mixtures of propylene glycol and 1,4-butanediol.

Very particularly preferred surface postcrosslinkers are 2-hydroxyethyl-2-oxazolidinone, 2-oxazolidinone and 1,3-propanediol.

In addition, it is also possible to use surface postcrosslinkers which comprise additional polymerizable ethylenically unsaturated groups, as described in DE 37 13 601 A1.

The amount of surface postcrosslinker is preferably 0.001 to 2% by weight, more preferably 0.02 to 1% by weight and most preferably 0.05 to 0.2% by weight, based in each case on the polymer particles.

In a preferred embodiment of the present invention, polyvalent cations are applied to the particle surface in addition to the surface postcrosslinkers before, during or after the surface postcrosslinking.

The polyvalent cations usable in the process according to the invention are, for example, divalent cations such as the cations of zinc, magnesium, calcium, iron and strontium, trivalent cations such as the cations of aluminum, iron, chromium, rare earths and manganese, tetravalent cations such as the cations of titanium and zirconium. Possible counterions are hydroxide, chloride, bromide, sulfate, hydrogensulfate, carbonate, hydrogencarbonate, nitrate, phosphate, hydrogenphosphate, dihydrogenphosphate and carboxylate, such as acetate, citrate and lactate. Salts with different counterions are also possible, for example basic aluminum salts such as aluminum monoacetate or aluminum monolactate. Aluminum sulfate, aluminum monoacetate and aluminum lactate are preferred. Apart from metal salts, it is also possible to use polyamines as polyvalent cations.

The amount of polyvalent cation used is, for example, 0.001 to 1.5% by weight, preferably 0.005 to 1% by weight and more preferably 0.02 to 0.8% by weight, based in each case on the polymer particles.

The surface postcrosslinking is typically performed in such a way that a solution of the surface postcrosslinker is sprayed onto the dried polymer particles. After the spray application, the polymer particles coated with surface postcrosslinker are dried thermally, and the surface postcrosslinking reaction can take place either before or during the drying.

The spray application of a solution of the surface postcrosslinker is preferably performed in mixers with moving mixing tools, such as screw mixers, disk mixers and paddle mixers. Particular preference is given to horizontal mixers such as paddle mixers, very particular preference to vertical mixers. The distinction between horizontal mixers and vertical mixers is made by the position of the mixing shaft, i.e. horizontal mixers have a horizontally mounted mixing shaft and vertical mixers a vertically mounted mixing shaft. Suitable mixers are, for example, horizontal Pflugschar® plowshare mixers (Gebr. Lödige Maschinenbau GmbH; Paderborn; Germany), Vrieco-Nauta continuous mixers (Hosokawa Micron BV; Doetinchem; the Netherlands), Processall Mixmill mixers (Processall Incorporated; Cincinnati; USA) and Schugi Flexomix® (Hosokawa Micron BV; Doetinchem; the Netherlands). However, it is also possible to spray on the surface postcrosslinker solution in a fluidized bed.

The surface postcrosslinkers are typically used in the form of an aqueous solution. The penetration depth of the surface postcrosslinker into the polymer particles can be adjusted via the content of nonaqueous solvent and total amount of solvent.

When exclusively water is used as the solvent, a surfactant is advantageously added. This improves the wetting behavior and reduces the tendency to form lumps. However, preference is given to using solvent mixtures, for example isopropanol/water, 1,3-propanediol/water and propylene glycol/water, where the mixing ratio in terms of mass is preferably from 20:80 to 40:60.

The thermal drying is preferably carried out in contact driers, more preferably paddle driers, most preferably disk driers. Suitable driers are, for example, Hosokawa Bepex® Horizontal Paddle Dryers (Hosokawa Micron GmbH; Leingarten; Germany), Hosokawa Bepex® Disc Dryers (Hosokawa Micron GmbH; Leingarten; Germany), Holo-FMK) driers (Metso Minerals Industries Inc.; Danville; USA) and Nara Paddle Dryers (NARA Machinery Europe; Frechen; Germany). Moreover, fluidized bed driers may also be used.

The drying can be effected in the mixer itself, by heating the jacket or blowing in warm air. Equally suitable is a downstream drier, for example a shelf drier, a rotary tube oven or a heatable screw. It is particularly advantageous to effect mixing and drying in a fluidized bed drier.

Preferred drying temperatures are in the range of 100 to 250° C., preferably 120 to 220° C., more preferably 130 to 210° C. and most preferably 150 to 200° C. The preferred residence time at this temperature in the reaction mixer or drier is preferably at least 10 minutes, more preferably at least 20 minutes, most preferably at least 30 minutes, and typically at most 60 minutes.

In a preferred embodiment of the present invention, the water-absorbing polymer particles are cooled after the thermal drying. The cooling is preferably performed in contact coolers, more preferably paddle coolers and most preferably disk coolers. Suitable coolers are, for example, Hosokawa Bepex® Horizontal Paddle Cooler (Hosokawa Micron GmbH; Leingarten; Germany), Hosokawa Bepex® Disc Cooler (Hosokawa Micron GmbH; Leingarten; Germany), Holo-Flite® coolers (Metso Minerals Industries Inc.; Danville; U.S.A.) and Nara Paddle Cooler (NARA Machinery Europe; Frechen; Germany). Moreover, fluidized bed coolers may also be used.

In the cooler, the water-absorbing polymer particles are cooled to 20 to 150° C., preferably 30 to 120° C., more preferably 40 to 100° C. and most preferably 50 to 80° C.

Subsequently, the surface postcrosslinked polymer particles can be classified again, excessively small and/or excessively large polymer particles being removed and recycled into the process.

To further improve the properties, the surface postcrosslinked polymer particles can be coated or remoisturized.

The remoisturizing is preferably performed at 30 to 80° C., more preferably at 35 to 70° C., most preferably at 40 to 60° C. At excessively low temperatures, the water-absorbing polymer particles tend to form lumps, and, at higher temperatures, water already evaporates to a noticeable degree. The amount of water used for remoisturizing is preferably from 1 to 10% by weight, more preferably from 2 to 8% by weight and most preferably from 3 to 5% by weight. The remoisturizing increases the mechanical stability of the polymer particles and reduces their tendency to static charging. The remoisturizing is advantageously performed in the cooler after the thermal drying.

Suitable coatings for improving the free swell rate and the permeability (SFC) are, for example, inorganic inert substances, such as water-insoluble metal salts, organic polymers, cationic polymers and di- or polyvalent metal cations. Suitable coatings for dust binding are, for example, polyols. Suitable coatings for counteracting the undesired caking tendency of the polymer particles are, for example, fumed silica, such as Aerosil® 200, and surfactants, such as Span® 20.

The water-absorbing polymer particles have a centrifuge retention capacity (CRC) of typically at least 15 g/g, preferably at least 20 g/g, more preferably at least 22 g/g, especially preferably at least 24 g/g and most preferably at least 26 g/g. The centrifuge retention capacity (CRC) of the water-absorbing polymer particles is typically less than 60 g/g. The centrifuge retention capacity (CRC) is determined by EDANA recommended test method No. WSP 241.2-05 “Fluid Retention Capacity in Saline, After Centrifugation”.

The inventive mixtures preferably comprise at least 80% by weight, preferably at least 85% by weight, more preferably at least 90% by weight and most preferably at least 95% by weight of water-absorbing polymer particles.

The spherical activated carbon can be produced by pyrolysis of spherical organic material, for example polystyrene. However, it is also possible to pyrolyze glucose solutions, as described in Int. J. Electrochem. Sci., Vol. 4, 2009, pages 1063 to 1073. Suitable spherical activated carbons are also available as SARATECH® 100562, SARATECH® 100772 and SARATECH® 101373 (Blucher GmbH, Erkrath, Germany).

The spherical activated carbon has a surface area of preferably 10 to 10 000 m²/g, more preferably of 100 to 5000 m²/g, most preferably of 1000 to 2000 m²/g.

The mean particle size of the spherical activated carbon is preferably at least 300 μm, more preferably from 350 to 550 μm and very particularly from 400 to 500 μm. The mean particle size of the product fraction may be determined by means of EDANA recommended test method No. WSP 220.2-05 “Particle Size Distribution”, where the proportions by mass of the screen fractions are plotted in cumulated form and the mean particle size is determined graphically. The mean particle size here is the value of the mesh size which gives rise to a cumulative 50% by weight.

The proportion of spherical activated carbon having a particle size of 300 to 600 μm is preferably at least 90% by weight, more preferably at least 95% by weight, most preferably at least 98% by weight.

The inventive mixtures preferably comprise at least 0.1% by weight, more preferably at least 0.5% by weight, preferentially at least 1% by weight and most preferably at least 5% by weight of spherical activated carbon.

The method of mixing is not subject to any restriction and it can be done as early as in the course of production of the water-absorbing polymer particles, for example in the course of cooling after the surface postcrosslinking or the subsequent classification, or in a specific mixer. Suitable mixers have already been described above for the surface postcrosslinking of the water-absorbing polymer particles.

The present invention is based on the finding that spherical activated carbon has a high abrasion resistance and is present in the inventive mixtures in isolated form alongside the water-absorbing polymer particles. The occurrence of fine dust or staining of the water-absorbing polymer particles by abraded material is prevented.

The odor-inhibiting mixtures may additionally comprise metal peroxides, oxidases and/or zeolites.

The metal peroxide is preferably the peroxide of a metal of main group 1, of main group 2, and/or of transition group 2 of the Periodic Table of the Elements, more preferably the peroxide of a metal of transition group 2 of the Periodic Table of the Elements.

Suitable metal peroxides are, for example, lithium peroxide, strontium peroxide, barium peroxide, sodium peroxide, magnesium peroxide and calcium peroxide, more preferably zinc peroxide.

The inventive mixture comprises preferably 0.001 to 5% by weight, more preferably from 0.01 to 3% by weight, especially preferably from 0.1 to 1.5% by weight and most preferably from 0.2 to 0.8% by weight of the metal peroxide.

Metal peroxides, especially zinc peroxide, have good odor-inhibiting action, and the mixtures produced therewith have high storage stability.

The mixtures comprise preferably less than 1 ppm, more preferably less than 10 ppm and most preferably less than 5 ppm of heavy metal ions. Heavy metal ions, especially iron ions, lead to the catalytic decomposition of the metal peroxides and hence lower the storage stability of the mixtures.

Suitable zeolites are, for example, zeolites with cations of main group 1, of main group 2, of transition group 1 and/or of transition group 2 of the Periodic Table of the Elements.

Suitable cations are, for example, zinc cations, silver cations and copper cations, more preferably titanium cations.

The inventive mixture comprises preferably 0.001 to 5% by weight, more preferably from 0.01 to 3% by weight, especially preferably from 0.1 to 1.5% by weight and most preferably from 0.2 to 0.8% by weight of the zeolite.

Zeolites likewise have good odor-inhibiting action.

Suitable oxidases are oxidases of the EC 1.1.3.x group, such as glucose oxidases (EC number 1.1.3.4), of the EC 1.3.3.x group, such as bilirubin oxidases (EC number 1.3.3.5), of the EC 1.4.3.x group, such as glycine oxidases (EC number 1.4.3.19), of the EC 1.5.3.x group, such as polyamine oxidases (EC number 1.5.3.11), of the EC 1.6.3.x group, such as NAD(P)H oxidases (EC number 1.6.3.1), of the EC 1.7.3.x group, such as hydroxylamine oxidases (EC number 1.7.3.4), of the EC 1.8.3.x group, such as sulfite oxidases (EC number 1.8.3.1), of the EC 1.9.3.x group, such as cytochrome oxidases (EC number 1.9.3.1), of the EC 1.10.3.x group, such as catechol oxidases (EC number 1.10.3.1), of the EC 1.16.3.x group, such as ferroxidase (EC number 1.16.3.1), of the EC 1.17.3.x group, such as xanthine oxidases (EC number 1.17.3.2), and of the EC 1.21.3.z group, such as reticuline oxidases (EC number 1.21.3.3).

Advantageously, a glucose oxidase (EC number 1.1.3.4) is used. It is even more advantageous when the glucose oxidase comprises a very low level of or even no catalase at all (EC number 1.11.1.6).

The specific catalytic oxidase activity of the odor-inhibiting mixture is preferably from 0.01 to 1000 μmol of substrate g⁻¹·min⁻¹, more preferably from 0.1 to 100 μmol of substrate g⁻¹·min⁻¹, most preferably from 1 to 10 μmol of substrate g⁻¹·min⁻¹.

The specific catalytic oxidase activity of the mixture can be determined by customary methods. However, it is better to determine the catalytic activity of the oxidase itself and to calculate the specific catalytic oxidase activity of the mixture.

Oxidases can reduce unpleasant odors, more particularly unpleasant odors caused by sulfur compounds. This is possibly brought about by hydrogen peroxide produced as a result of the catalytic oxidase activity. Therefore, simultaneous use of peroxidases should be avoided.

The odor-inhibiting mixtures may additionally comprise the substrate of the oxidase. A substrate is a compound which is converted by the enzyme in a chemical reaction. The first step of an enzymatic reaction involves the formation of an enzyme-substrate complex which leads, after the reaction, to the release of product and enzyme, and so the catalytic cycle can be run through once again. An enzyme may possibly convert various substrates which are often chemically similar. Substrates in the context of the present invention are substrates of the oxidases usable in accordance with the invention, for example β-D-glucose for glucose oxidase.

Preferably from 0.5 to 25% by weight, more preferably from 5 to 20% by weight and most preferably from 8 to 15% by weight of the substrate is used, based in each case on the water-absorbing polymer particles.

The substrates can also be used in encapsulated form, such that the oxidase is not available until liquid is added, for example by virtue of a coating with water-soluble polymers such as polyvinyl alcohol. However, it is also possible, instead or additionally, to encapsulate the oxidases for use in accordance with the invention.

The present invention further provides hygiene articles comprising an inventive mixture, especially hygiene articles for light and heavy incontinence.

The hygiene articles typically comprise a water-impervious backside, a water-pervious topside and an intermediate absorbent core composed of the inventive water-absorbing polymer particles and fibers, preferably cellulose. The proportion of the inventive water-absorbing polymer particles in the absorbent core is preferably 20 to 100% by weight and more preferably 50 to 100% by weight.

### Methods:

The standard test methods described hereinafter and designated “WSP” are described in: “Standard Test Methods for the Nonwovens Industry”, 2005 edition, published jointly by the Worldwide Strategic Partners EDANA (Avenue Eugène Plasky, 157, 1030 Brussels, Belgium, www.edana.org) and INDA (1100 Crescent Green, Suite 115, Cary, N.C. 27518, USA, www.inda.org). This publication is available both from EDANA and from INDA.

The measurements should, unless stated otherwise, be conducted at an ambient temperature of 23±2° C. and a relative air humidity of 50±10%. The water-absorbing polymer particles are mixed thoroughly before the measurement.

### Residual Monomers

The residual monomer content of the water-absorbing polymer particles is determined by EDANA recommended test method WSP No. 210.2-05 “Residual Monomers”.

### Moisture Content

The moisture content of the water-absorbing polymer particles is determined by EDANA recommended test method No. WSP 230.2-05 “Mass Loss Upon Heating”.

### Centrifuge Retention Capacity

The centrifuge retention capacity (CRC) is determined by EDANA recommended test method No. WSP 241.2-05 “Fluid Retention Capacity in Saline, After Centrifugation”.

Absorption Under a Pressure of 49.2 g/cm² (Absorption Under Load)

The absorption under a pressure of 49.2 g/cm² (AUL0.7 psi) is determined analogously to EDANA recommended test method No. WSP 242.2-05 “Absorption Under Pressure, Gravimetric Determination”, except that a pressure of 49.2 g/cm² (AUL0.7 psi) is established instead of a pressure of 21.0 g/cm² (AUL0.3 psi).

### CIE Color Number (L, a, b)

The color analysis is carried out according to the CIELAB method (Hunterlab, volume 8, 1996, book 7, pages 1 to 4) with a “LabScan XE S/N LX17309” colorimeter (HunterLab, Reston, US). This method describes the colors via the coordinates L, a and b of a three-dimensional system. L indicates the brightness, where L=0 means black and L=100 white. The values of a and b indicate the positions of the color on the red/green and yellow/blue color axes respectively, where +a represents red, −a green, +b yellow and −b blue. The HC60 is calculated by the formula HC60=L−3b.

The color measurement corresponds to the three-area method according to DIN 5033-6.

### EXAMPLES

### Production of the Water-Absorbing Polymer Particles

### Example 1

25.1 kg of sodium acrylate (37.5% by weight solution in water) and 2.9 kg of acrylic acid were mixed with 19 g of 15-tuply ethoxylated trimethylolpropane triacrylate. The initiator used was a 15% by weight aqueous solution of 2,2′-azobis[2-(2-imidazolin-2-yl)propane]dihydrochloride and a 15% by weight aqueous solution of sodium peroxodisulfate. The initiators were metered into the monomer solution by means of a static mixer upstream of a dropletizer. The dropletizer plate had 20×200 μm holes. The resulting mixture was dropletized into a heated dropletization tower filled with a nitrogen atmosphere (height 12 m, width 2 m, gas velocity 0.27 m/s in cocurrent). The metering rate of the monomer solution was 28 kg/h. The metering rate of each of the initiator solutions was 0.23 kg/h. The heating output of the gas preheating was regulated such that the gas outlet temperature in the dropletization tower was constant at 124° C.

The water-absorbing polymer particles were subsequently analyzed. The residual monomer content was 4500 ppm, the moisture content 5.7% by weight, the centrifuge retention capacity (CRC) 33.7 g/g and the absorption under pressure (AUL0.7 psi) 22.7 g/g.

### Production of the Mixtures

### Example 2

### Noninventive

270 g of water-absorbing polymer particles from example 1 and 13.5 g of activated carbon of the Acticarbone 3S type (CECA, La Garenne Colombes, France) were weighed into a 500 ml square plastic bottle. This mixture was homogenized in a tumbling mixer at 49 rpm for 15 minutes and analyzed. The results are summarized in table 1 (sample A).

Subsequently, the 500 ml square plastic bottle was emptied and not cleaned. Another 270 g of water-absorbing polymer particles from example 1 were weighed in, but no activated carbon. This mixture was likewise homogenized in a tumbling mixer at 49 rpm for 15 minutes and analyzed. The results are summarized in table 1 (sample B).

### Example 3

270 g of water-absorbing polymer particles from example 1 and 13.5 g of spherical activated carbon of the SARATECH® 100562 type (Blücher GmbH, Erkrath, Germany) were weighed into a 500 ml square plastic bottle. This mixture was homogenized in a tumbling mixer at 49 rpm for 15 minutes and analyzed. The results are summarized in table 1 (sample A).

Subsequently, the 500 ml square plastic bottle was emptied and not cleaned. Another 270 g of water-absorbing polymer particles from example 1 were weighed in, but no activated carbon. This mixture was likewise homogenized in a tumbling mixer at 49 rpm for 15 minutes and analyzed. The results are summarized in table 1 (sample B).

TABLE 1
Addition of different activated carbons

| | Sample | L | a | b | HC60 |
|---|---|---|---|---|---|
| Example 1*) | | 88.62 | −1.76 | 15.10 | 43.33 |
| Example 2*) | A | 13.72 | 0.08 | 0.66 | 11.73 |
| | B | 63.70 | −0.90 | 5.74 | 46.48 |
| Example 3 | A | 66.96 | −1.39 | 5.25 | 51.22 |
| | B | 88.33 | −1.85 | 14.55 | 44.69 |

*) noninventive

### Example 4

### Noninventive

270 g of water-absorbing polymer particles (HySorb® B7055; BASF SE; Germany) and 13.5 g of activated carbon of the Acticarbone 3S type (CECA, La Garenne Colombes, France) were weighed into a 500 ml square plastic bottle. This mixture was homogenized in a tumbling mixer at 49 rpm for 15 minutes and analyzed. The results are summarized in table 2 (sample A).

Subsequently, the 500 ml square plastic bottle was emptied and not cleaned. Another 270 g of water-absorbing polymer particles were weighed in, but no activated carbon. This mixture was likewise homogenized in a tumbling mixer at 49 rpm for 15 minutes and analyzed. The results are summarized in table 2 (sample B).

### Example 5

270 g of water-absorbing polymer particles (HySorb® B7055; BASF SE; Germany) and 13.5 g of spherical activated carbon of the SARATECH® 100562 type (Blucher GmbH, Erkrath, Germany) were weighed into a 500 ml square plastic bottle. This mixture was homogenized in a tumbling mixer at 49 rpm for 15 minutes and analyzed. The results are summarized in table 2 (sample A).

Subsequently, the 500 ml square plastic bottle was emptied and not cleaned. Another 270 g of water-absorbing polymer particles were weighed in, but no activated carbon. This mixture was likewise homogenized in a tumbling mixer at 49 rpm for 15 minutes and analyzed. The results are summarized in table 2 (sample B).

TABLE 2
Addition of different activated carbons to HySorb ® B7055

| | Sample | L | a | b | HC60 |
|---|---|---|---|---|---|
| HySorb ® B7055 | | 93.10 | −1.00 | 5.67 | 76.09 |
| Example 4*) | A | 14.47 | 0.12 | 0.37 | 13.35 |
| | B | 58.78 | 0.06 | 1.54 | 54.17 |
| Example 5 | A | 63.33 | −0.02 | 0.80 | 60.92 |
| | B | 89.83 | −0.95 | 5.20 | 74.24 |

*) noninventive

Examples 4 and 5 were conducted with Hysorb® B7055 (BASF SE; Ludwigshafen; Germany), commercial surface postcrosslinked water-absorbing polymer particles based on sodium acrylate with a neutralization level of 70 mol %.

Such surface postcrosslinked water-absorbing polymer particles are commercially available, for example, from BASF Aktiengesellschaft (trade name: HySorb®), from Stockhausen GmbH (trade name: Favor®) and from Nippon Shokubai Co., Ltd. (trade name: Aqualic®).

1. An odor-inhibiting mixture comprising water-absorbing polymer particles and spherical activated carbon.

2. The mixture according to claim 1, comprising at least 80% by weight of the water-absorbing polymer particles.

3. The mixture according to claim 1, wherein the water-absorbing polymer particles have a mean particle size of 250 to 600 μm.

4. The mixture according to claim 1, wherein at least 90% by weight of the water-absorbing polymer particles have a particle size of 150 to 850 μm.

5. The mixture according to claim 1, wherein the water-absorbing polymer particles have a centrifuge retention capacity of at least 15 g/g.

6. The mixture according to claim 1, comprising at least 1% by weight of the spherical activated carbon.

7. The mixture according to claim 1, wherein the spherical activated carbon has a mean particle size of 350 to 550 μm.

8. The mixture according to claim 1, wherein at least 90% by weight of the spherical activated carbon has a particle size of 300 to 600 μm.

9. The mixture according to claim 1, wherein the spherical activated carbon has a surface area of 10 to 10 000 m²/g.

10. The mixture according to claim 1, which additionally comprises a metal peroxide.

11. The mixture according to claim 10, wherein the metal peroxide is zinc peroxide.

12. The mixture according to claim 1, which additionally comprises an oxidase.

13. The mixture according to claim 12, wherein the oxidase is glucose oxidase.

14. The mixture according to claim 13, which additionally comprises glucose.

15. A hygiene article comprising, a mixture according to claim 1.
